(19) United States
(12) Patent Application Publication
(10) Pub. No.: US 2020/0337993 A1
(43) Pub. Date: Oct. 29, 2020

(54) INTRACANALICULAR HYDROGEL INSERTS FOR THE DELIVERY OF ANESTHETICS

(71) Applicant: Ocular Therapeutix, Inc., Bedford, MA (US)

(72) Inventors: Peter Jarrett, Burlington, MA (US); Michael Goldstein, Cambridge, MA (US); Rami El-Hayek, Norwood, MA (US); Timothy S. Jarrett, Boston, MA (US)

(21) Appl. No.: 16/857,458

(22) Filed: Apr. 24, 2020

Related U.S. Application Data
(60) Provisional application No. 62/838,789, filed on Apr. 25, 2019.

(51) Int. Cl. A61K 9/00; A61K 9/06; A61K 47/34; A61K 31/445; A61P 27/02

(52) U.S. Cl. CPC A61K 9/0051; A61K 9/06; A61P 27/02; A61K 31/445; A61K 47/34

(57) ABSTRACT

Provided herein are sustained-release biodegradable ocular hydrogel inserts which are useful in the treatment of certain ocular conditions.

### RELATED APPLICATIONS

This application claims priority to U.S. Provisional Application No. 62/838,789, filed Apr. 25, 2019, the entire contents of which are incorporated herein by reference.

### BACKGROUND

Trauma to the eye, particularly corneal injury or abrasion, is a common injury that can be extremely painful. Although ocular anesthetics such as bupivacaine (BPI), proparacaine, and teracaine are commonly used in clinical settings, these agents are typically administered as eye drops and have rapid onsets of action (0.25 to 10 minutes) and a limited duration of action (up to 30 minutes). In addition, the concentrations of these agents needed to achieve corneal anesthesia is between 0.25% to 4%. At these concentrations, ocular anesthetics can cause the development of temporary superficial corneal epithelial lesions. Upon repeated use, either in frequency or length of time, these lesions progress to extensive erosions of the corneal epithelium and grayish infiltrates of the corneal stroma. This can lead to permanent scarring and loss of vision. Prolonged application of ocular anesthetics is further associated with delayed corneal reepithelialization after wounding, altered lacrimation, corneal swelling, and disruption of epithelial cell mitosis and migration.

The short duration and toxicity concerns with current ocular anesthetics preclude their widespread use for chronic pain conditions as well as for lengthier ophthalmic clinical procedures. Additionally, physicians are reluctant to allow patients the option to self-administer ocular anesthetics because of toxicity concerns associated with overuse.

A more safe and effective formulation comprising one or more ophthalmic anesthetics is clinically needed in ophthalmology for longer duration pain management.

### SUMMARY

Provided herein are safe and effective hydrogel compositions which allow for the sustained release of one or more ocular anesthetics. Also provided is the use of these hydrogel compositions in the treatment or prevention of ocular discomfort such as ocular pain.

The disclosed compositions effectively delivered therapeutic amounts of the anesthetic bupivacaine to male beagle dogs with corneal wounds over the course of about 5 days, and substantially reduced corneal sensation. See e.g., Table 4 showing that elevated concentrations of bupivacaine were present in the tear fluid for 4 days followed by a steady decline beginning at day 5. No substantial difference in the rate of corneal wound healing was observed between treated and untreated dogs.

The disclosed compositions had no negative impact in the rate of corneal wound healing between eyes treated with an inventive composition comprising bupivacaine and untreated controls. See FIG. 6. In addition, no negative effects on the overall general health of the animals were observed using intracanalicular administration of a disclosed composition comprising bupivacaine.

### BRIEF DESCRIPTION OF THE FIGURES

FIG. 1A illustrates a schematic of the dispersion of anesthetic and outer clearance zone of one aspect of the disclosed hydrogel composition.

FIG. 1B shows the dispersion of anesthetic and outer clearance zone of an inventive hydrogel composition.

FIG. 2 illustrates the in vitro release of bupivacaine using an inventive composition.

FIG. 3 shows the average corneal sensation scores between treated and non-treated beagle dogs.

FIG. 4 shows the combined average corneal sensation scores between treated and non-treated beagle dogs.

FIG. 5 shows fluorescein stating of wounded corneal tissue over time in untreated control and Inventive Composition treated eyes of male beagle dogs.

FIG. 6 shows wound corneal tissue area over time in untreated control and Inventive Composition treated eyes of male beagle dogs as a percentage over baseline.

### DETAILED DESCRIPTION

Provided herein are ocular hydrogel compositions comprising an anesthetic and a polymer network, wherein the anesthetic is delivered over an extended period of time (e.g., 12 hours or longer).

Also provided herein are methods, uses, and medicament formulations for treating or preventing ocular discomfort in a subject, comprising administering to the eye of the subject a therapeutically effective amount of the ocular hydrogel composition.

Further provided are processes for preparing a disclosed ocular hydrogel composition.

### 1. Definitions

The term “biodegradable” refers to a material, such as the disclosed ocular hydrogel compositions, which degrade in vivo. Degradation of the material occurs over time and may occur concurrently with, or subsequent to, release of the anesthetic. In one aspect, “biodegradable” means that complete dissolution of the ocular composition occurs, i.e., there is no residual compositional matter remaining e.g., in the eye of a subject. In an alternative aspect, degradation may occur independently of anesthetic release such that e.g., residual composition matter remains following degradation.

The term “polymer network” refers to a group of polymers comprising multiple branch structures (arms) cross-linked to other polymer chains. The polymer chains may be of the same or different chemical structures, e.g., as in complementary or non-complementary repeating units.

Nomenclature for synthetic precursors used to generate the disclosed polymer networks are referenced using the number of arms followed by the MW of the PEG and then the reactive group (e.g., electrophile or nucleophile). For example 4a20K PEG SAZ refers to a 20,000 Da PEG with 4 arms with a succinimidylazelate end group, 4a20K PEG SAP refers to a 20,000 Da PEG with 4 arms with a succinimidyladipate end group, 4a20K PEG SG refers to a 20,000 Da PEG with 4 arms with a succinimidylglutarate end group, 4a20K PEG SS refers to a 20,000Da PEG with 4 arms with a succinimidylsuccinate end group, etc. Similarly, 4a20K PEG NH2 means a 20,000 Da PEG with 4 arms with an amine end group, 8a20K PEG NH2 means a 20,000 Da PEG with 8 arms with an amine end group, etc.

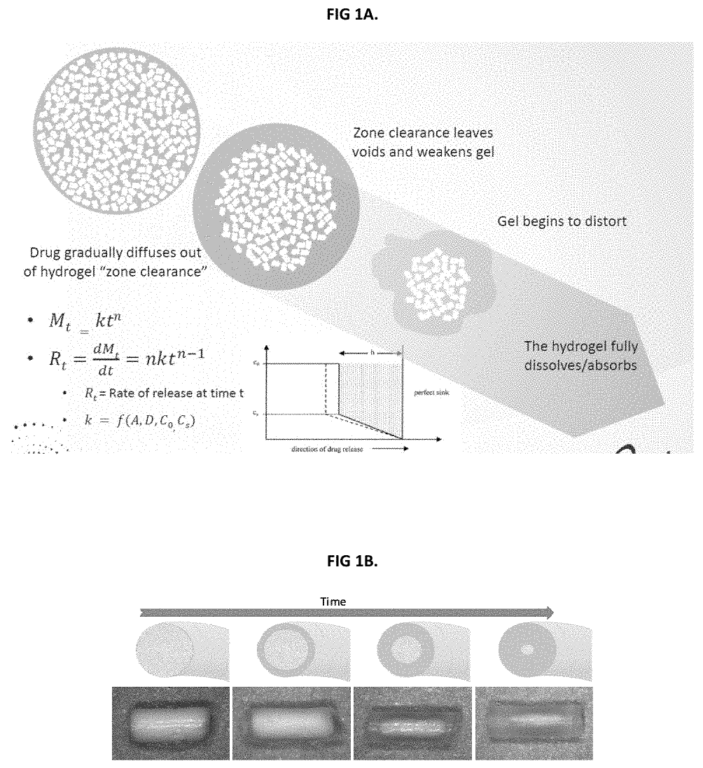

The term “clearance zone” refers to a portion of the hydrogel which is devoid of undissolved anesthetic particles prior to, or following the release of the anesthetic. “Clearance zone” and “zone clearance” are used interchangeably. An exemplary representation of the clearance zone is depicted in FIG. 1. As shown, the clearance zone provides a protective barrier between the undissolved anesthetic (e.g., undissolved anesthetic) comprised in the hydrogel composition and the adjacent tissue in the eye. Without wishing to be bound by theory, this is because the surface concentration is limited to the solubility of the anesthetic in water. As the properties of the polymer network change, e.g., as the polymer network slowly degrades, anesthetic continues to be released from the hydrogel composition by first passing through the clearance zone before it is released and comes in direct contact with the eye. In one aspect, the release of the anesthetic is solubility driven and is not affected by polymer network changes, except for dimensional changes that accompany polymer changes. In some aspects, the overall size of the clearance zone increases as more anesthetic is released from the hydrogel composition. In one aspect, there is a desire to match the size of the clearance zone and the rate of degradation of the hydrogel composition. For example, the polymer hydrolysis rate is matched to the anesthetic solubility so that as the size of the clearance zone increases, the hydrogel degradation increases so that hydrogel disappearance coincides roughly with anesthetic disappearance.

The term “amorphous” refers to a polymer or polymer network which does not exhibit crystalline structures in X-ray or electron scattering experiments.

The term “semi-crystalline” refers to a polymer or polymer network which possesses some crystalline character, i.e., exhibits crystalline properties in thermal analysis, X-ray scattering or electron scattering experiments. In some aspects, “semi-crystalline” polymers or networks of polymers have a highly ordered molecular structure with sharp melt points. In some aspects, “semi-crystalline” polymers or networks of polymers do not gradually soften with a temperature increase and instead remain solid until a given quantity of heat is absorbed and then rapidly change into a rubber or liquid.

As used herein, “homogenously dispersed” means the component, such as the anesthetic, is uniformly dispersed throughout the hydrogel or polymer network, except for the portion comprising the clearance zone.

The term “treat”, “treating”, or “treatment” are used interchangeably and refer to reversing, alleviating, delaying the onset of, or inhibiting the progress of ocular discomfort, or one or more symptoms thereof, as described herein.

The term “preventing”, “prevention”, or “prevent” are used interchangeably and include the prevention of the recurrence, spread, or onset of a disclosed ocular discomfort. Prevention also includes the administration of provided composition in order to induce insensitivity to pain prior to the occurrence of ocular discomfort, e.g., to induce insensitivity prior to a surgical or non-invasive procedure on the eye.

The terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

The term “effective amount” or “therapeutically effective amount” refers to an amount of a disclosed composition that will elicit a biological or medical response of a subject. It will be understood that the specific dosage and treatment regimen for any particular patient will depend upon a variety of factors, including the activity of the specific protein employed, the age, body weight, general health, sex, diet, time of administration, rate of excretion, the judgment of the treating physician and the severity of the particular condition being treated or prevented.

### 2. Compositions

As part of a first embodiment, provided herein is a biodegradable hydrogel composition comprising an anesthetic and a polymer network, wherein said anesthetic is delivered to the eye in a sustained manner for a period of about 12 hours or longer.

As part of a second embodiment, the polymer network of the disclosed hydrogel composition (e.g., as in the first embodiment) comprises a plurality of polyethylene glycol (PEG) units. Alternatively, as part of a second embodiment, the polymer network of the disclosed hydrogel composition (e.g., as in the first embodiment) comprises a plurality of multi-arm PEG units.

As part of a third embodiment, the plurality of polyethylene glycol (PEG) units included in the disclosed compositions are cross-linked to form a polymer network comprising a plurality of multi-arm PEG units having at least 2 arms, wherein the remaining features of the compositions are described herein e.g., as in the first or second embodiment. Alternatively, as part of a third embodiment, the polymer network of the disclosed compositions comprise a plurality of multi-arm PEG units having from 2 to 10 arms, wherein the remaining features of the compositions are described herein e.g., as in the first or second embodiment. In another alternative, as part of a third embodiment, the polymer network of the disclosed compositions comprise a plurality of multi-arm PEG units having from 4 to 8 arms, wherein the remaining features of the compositions are described herein e.g., as in the first or second embodiment. In another alternative, as part of a third embodiment, the polymer network of the disclosed compositions comprise a plurality of 4-arm PEG units, wherein the remaining features of the compositions are described herein e.g., as in the first or second embodiment. In another alternative, as part of a third embodiment, the polymer network of the disclosed compositions comprise a plurality of 8-arm PEG units, wherein the remaining features of the compositions are described herein e.g., as in the first or second embodiment.

As part of a fourth embodiment, the polymer network of the disclosed compositions comprises a plurality of PEG units having a number average molecular weight (Mn) ranging from about 5 KDa to about 50 KDa, wherein the remaining features of the compositions are described herein e.g., as in the first through third embodiments. Alternatively, as part of a fourth embodiment, the polymer network of the disclosed compositions comprises a plurality of PEG units having a number average molecular weight (Mn) ranging from about 5 KDa to about 40 KDa, wherein the remaining features of the compositions are described herein e.g., as in the first through third embodiments. In another alternative, as part of a fourth embodiment, the polymer network of the disclosed compositions comprise a plurality of PEG units having a number average molecular weight (Mn) ranging from about 5 KDa to about 30 KDa, wherein the remaining features of the compositions are described herein e.g., as in the first through third embodiments. In another alternative, as part of a fourth embodiment, the polymer network of the disclosed compositions comprise a plurality of PEG units having a number average molecular weight (Mn) ranging from about 10 KDa to about 50 KDa, wherein the remaining features of the compositions are described herein e.g., as in the first through third embodiments. In another alternative, as part of a fourth embodiment, the polymer network of the disclosed compositions comprise a plurality of PEG units having a number average molecular weight (Mn) ranging from about 10 KDa to about 40 KDa, wherein the remaining features of the compositions are described herein e.g., as in the first through third embodiments. In another alternative, as part of a fourth embodiment, the polymer network of the disclosed compositions comprise a plurality of PEG units having a number average molecular weight (Mn) ranging from about 10 KDa to about 30 KDa, wherein the remaining features of the compositions are described herein e.g., as in the first through third embodiments. In another alternative, as part of a fourth embodiment, the polymer network of the disclosed compositions comprise a plurality of PEG units having a number average molecular weight (Mn) ranging from about 10 KDa to about 20 KDa, wherein the remaining features of the compositions are described herein e.g., as in the first through third embodiments. In another alternative, as part of a fourth embodiment, the polymer network of the disclosed compositions comprise a plurality of PEG units having a number average molecular weight (Mn) ranging from about 30 KDa to about 50 KDa, wherein the remaining features of the compositions are described herein e.g., as in the first through third embodiments. In another alternative, as part of a fourth embodiment, the polymer network of the disclosed compositions comprise a plurality of PEG units having a number average molecular weight (Mn) ranging from about 35 KDa to about 45 KDa, wherein the remaining features of the compositions are described herein e.g., as in the first through third embodiments. In another alternative, as part of a fourth embodiment, the polymer network of the disclosed compositions comprise a plurality of PEG units having a number average molecular weight (Mn) ranging from about 15 KDa to about 30 KDa, wherein the remaining features of the compositions are described herein e.g., as in the first through third embodiments. In another alternative, as part of a fourth embodiment, the polymer network of the disclosed compositions comprise a plurality of PEG units having a number average molecular weight (Mn) ranging from about 15 KDa to about 25 KDa, wherein the remaining features of the compositions are described herein e.g., as in the first through third embodiments. In another alternative, as part of a fourth embodiment, the polymer network of the disclosed compositions comprise a plurality of PEG units having a number average molecular weight (Mn) of at least about 5 KDa, wherein the remaining features of the compositions are described herein e.g., as in the first through third embodiments. In another alternative, as part of a fourth embodiment, the polymer network of the disclosed compositions comprise a plurality of PEG units having a number average molecular weight (Mn) of at least about 10 KDa, wherein the remaining features of the compositions are described herein e.g., as in the first through third embodiments. In another alternative, as part of a fourth embodiment, the polymer network of the disclosed compositions comprise a plurality of PEG units having a number average molecular weight (Mn) of at least 15 about KDa, wherein the remaining features of the compositions are described herein e.g., as in the first through third embodiments. In another alternative, as part of a fourth embodiment, the polymer network of the disclosed compositions comprise a plurality of PEG units having a number average molecular weight (Mn) of at least 20 about KDa, wherein the remaining features of the compositions are described herein e.g., as in the first through third embodiments. In another alternative, as part of a fourth embodiment, the polymer network of the disclosed compositions comprise a plurality of PEG units having a number average molecular weight (Mn) of at least 30 about KDa, wherein the remaining features of the compositions are described herein e.g., as in the first through third embodiments. In another alternative, as part of a fourth embodiment, the polymer network of the disclosed compositions comprise a plurality of PEG units having a number average molecular weight (Mn) of at least 40 about KDa, wherein the remaining features of the compositions are described herein e.g., as in the first through third embodiments. In another alternative, as part of a fourth embodiment, the polymer network of the disclosed compositions comprise a plurality of PEG units having a number average molecular weight (Mn) of about 10 KDa, wherein the remaining features of the compositions are described herein e.g., as in the first through third embodiments. In another alternative, as part of a fourth embodiment, the polymer network of the disclosed compositions comprise a plurality of PEG units having a number average molecular weight (Mn) of about 15 KDa, wherein the remaining features of the compositions are described herein e.g., as in the first through third embodiments. In another alternative, as part of a fourth embodiment, the polymer network of the disclosed compositions comprise a plurality of PEG units having a number average molecular weight (Mn) of about 20 KDa, wherein the remaining features of the compositions are described herein e.g., as in the first through third embodiments. In another alternative, as part of a fourth embodiment, the polymer network of the disclosed compositions comprise a plurality of PEG units having a number average molecular weight (Mn) of about 40 KDa, wherein the remaining features of the compositions are described herein e.g., as in the first through third embodiments.

In a fifth embodiment, the polymer network of the disclosed compositions comprise a plurality of PEG units crosslinked by a hydrolyzable linker, wherein the remaining features of the compositions are described herein e.g., as in the first through fourth embodiments. Alternatively, as part of a fifth embodiment, the polymer network of the disclosed compositions comprise a plurality of PEG units crosslinked by a hydrolyzable linker having the formula:

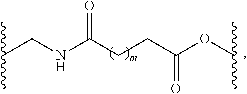

wherein m is an integer from 1 to 9, wherein the remaining features of the compositions are described herein e.g., as in the first through fourth embodiments. In another alternative, as part of a fifth embodiment, the polymer network of the disclosed compositions comprise a plurality of PEG units crosslinked by a hydrolyzable linker having the formula:

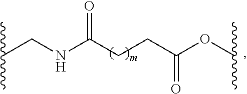

wherein m is an interger from 2 to 6 and wherein the remaining features of the compositions are described herein e.g., as in the first through fourth embodiments. In another alternative, as part of a fifth embodiment, the polymer network of the disclosed compositions comprise a plurality of PEG units having the formula:

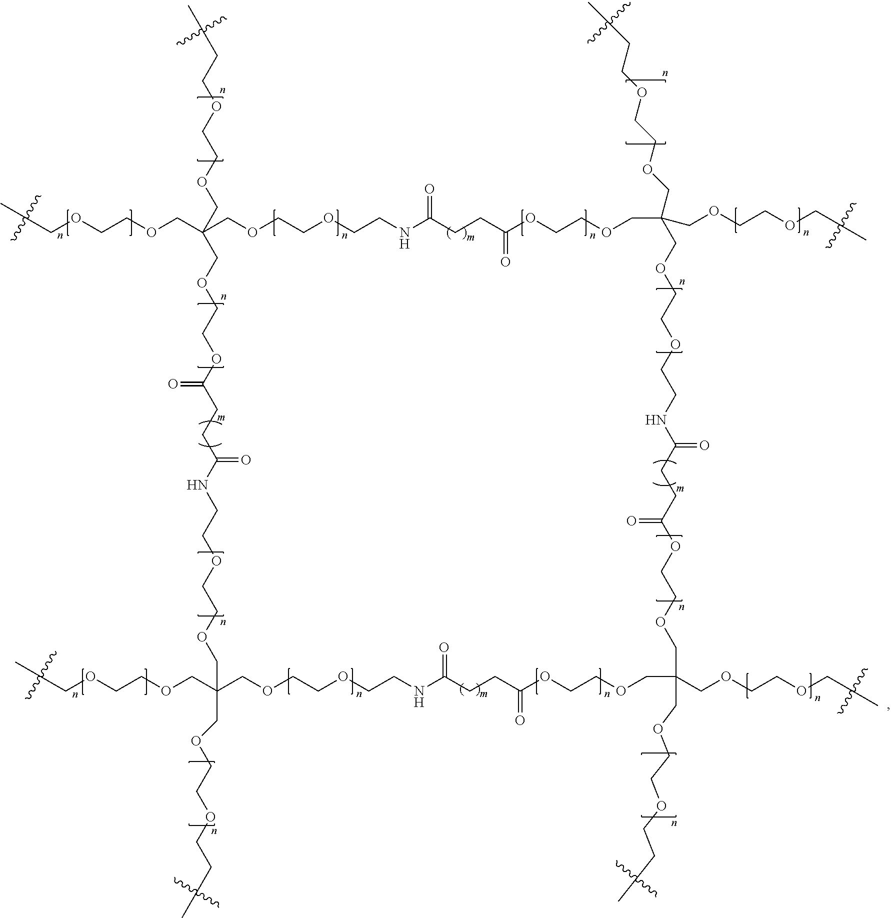

wherein n represents an ethylene oxide repeating unit and the dashed lines represent the points of repeating units of the polymer network, wherein the remaining features of the compositions are described herein e.g., as in the first through fourth embodiments. In another alternative, as part of a fifth embodiment, the polymer network of the disclosed compositions comprise a plurality of PEG units having the formula set forth above, but with an 8-arm PEG scaffold, wherein the remaining features of the compositions are described herein e.g., as in the first through fourth embodiments.

In a sixth embodiment, the polymer network of the disclosed compositions is formed by reacting a plurality of polyethylene glycol (PEG) units comprising groups which are susceptible to nucleophilic attack with one or more nucleophilic groups to form the polymer network, wherein the remaining features of the compositions are described herein e.g., as in the first through fifth embodiments. Examples of suitable groups which are susceptible to nucleophilic attack include, but art not limited to activated esters (e.g., thioesters, succinimidyl esters, benzotriazolyl esters, esters of acrylic acids, and the like). Examples of suitable nucleophilic groups include, but art not limited to, amines and thiols.

In a seventh embodiment, the polymer network of the disclosed compositions is formed by reacting a plurality of polyethylene glycol (PEG) units, each having a molecule weight as described above in the fourth embodiment and which comprise groups which are susceptible to nucleophilic attack, with one or more nucleophilic groups to form the polymer network, wherein the remaining features of the compositions are described herein e.g., as in the first through sixth embodiments. Alternatively, as part of a seventh embodiment, the polymer network of the disclosed hydrogel implant is formed by reacting a plurality of polyethylene glycol (PEG) units, each having a molecule weight as described above in the fourth embodiment and which comprise a succinimidyl ester group, with one or more nucleophilic groups to form the polymer network, wherein the remaining features of the compositions are described herein e.g., as in the first through fourth embodiments. In another alternative, as part of a seventh embodiment, the polymer network of the disclosed hydrogel implant is formed by reacting a plurality of polyethylene glycol (PEG) units selected from 4a20K PEG SAZ, 4a20K PEG SAP, 4a20K PEG SG, 4a20K PEG SS, 8a20K PEG SAZ, 8a20K PEG SAP, 8a20K PEG SG, 8a20K PEG SS, wherein the remaining features of the compositions are described herein e.g., as in the first through sixth embodiments.

In an eighth embodiment, the polymer network of the disclosed compositions is formed by reacting a plurality of polyethylene glycol (PEG) units comprising groups which are susceptible to nucleophilic attack with one or more amine groups to form the polymer network, wherein the remaining features of the compositions are described herein e.g., as in the first through seventh embodiments. Alternatively, as part of an eighth embodiment, the polymer network of the disclosed hydrogel implant is formed by reacting a plurality of polyethylene glycol (PEG) units comprising groups which are susceptible to nucleophilic attack with one or more PEG or Lysine based-amine groups to form the polymer network, wherein the remaining features of the compositions are described herein e.g., as in the first through seventh embodiments. In another alternative, as part of an eighth embodiment, the polymer network of the disclosed hydrogel implant is formed by reacting a plurality of polyethylene glycol (PEG) units comprising groups which are susceptible to nucleophilic attack with one or more PEG or Lysine based-amine groups selected from 4a20K PEG NH2, 8a20K PEG NH2, and trilysine, or salts thereof, wherein the remaining features of the compositions are described herein e.g., as in the first through seventh embodiments.

As part of a ninth embodiment, the polymer network of the disclosed compositions are amorphous (e.g., under aqueous conditions such as in vivo), wherein the remaining features of the compositions are described herein e.g., as in the first through eighth embodiments. Alternatively, as part of a ninth embodiment, the polymer network of the disclosed compositions are semi-crystalline (e.g., in the absence of water), wherein the remaining features of the compositions are described herein e.g., as in the first through eighth embodiments.

As part of a tenth embodiment, the anesthetic inhibitor of the disclosed compositions are homogenously dispersed (e.g., as a particulate) within the polymer network, wherein the remaining features of the compositions are described herein e.g., as in the first through ninth embodiments.

As part of an eleventh embodiment, the anesthetic of the disclosed compositions is delivered to the eye in a sustained manner for a period ranging from about 6 hours to about 20 days, wherein the remaining features of the compositions are described herein e.g., as in the first through tenth embodiments. Alternatively as part of an eleventh embodiment, the anesthetic of the disclosed compositions is delivered to the eye in a sustained manner for a period ranging from about 12 hours to about 20 days, wherein the remaining features of the compositions are described herein e.g., as in the first through tenth embodiments. In another alternative, as part of an eleventh embodiment, the anesthetic of the disclosed compositions is delivered to the eye in a sustained manner for a period ranging from about 12 hours to about 15 days, wherein the remaining features of the compositions are described herein e.g., as in the first through tenth embodiments. In another alternative, as part of an eleventh embodiment, the anesthetic of the disclosed compositions is delivered to the eye in a sustained manner for a period ranging from about 12 hours to about 10 days, wherein the remaining features of the compositions are described herein e.g., as in the first through tenth embodiments. In another alternative, as part of an eleventh embodiment, the anesthetic of the disclosed compositions is delivered to the eye in a sustained manner for a period ranging from about 12 hours to about 9 days, wherein the remaining features of the compositions are described herein e.g., as in the first through tenth embodiments. In another alternative, as part of an eleventh embodiment, the anesthetic of the disclosed compositions is delivered to the eye in a sustained manner for a period ranging from about 12 hours to about 8 days, wherein the remaining features of the compositions are described herein e.g., as in the first through tenth embodiments. In another alternative, as part of an eleventh embodiment, the anesthetic of the disclosed compositions is delivered to the eye in a sustained manner for a period ranging from about 12 hours to about 7 days, wherein the remaining features of the compositions are described herein e.g., as in the first through tenth embodiments. In another alternative, as part of an eleventh embodiment, the anesthetic of the disclosed compositions is delivered to the eye in a sustained manner for a period ranging from about 12 hours to about 6 days, wherein the remaining features of the compositions are described herein e.g., as in the first through tenth embodiments. In another alternative, as part of an eleventh embodiment, the anesthetic of the disclosed compositions is delivered to the eye in a sustained manner for a period ranging from about 12 hours to about 5 days, wherein the remaining features of the compositions are described herein e.g., as in the first through tenth embodiments. In another alternative, as part of an eleventh embodiment, the anesthetic of the disclosed compositions is delivered to the eye in a sustained manner for a period ranging from about 12 hours to about 4 days, wherein the remaining features of the compositions are described herein e.g., as in the first through tenth embodiments. In another alternative, as part of an eleventh embodiment, the anesthetic of the disclosed compositions is delivered to the eye in a sustained manner for a period ranging from about 12 hours to about 3 days, wherein the remaining features of the compositions are described herein e.g., as in the first through tenth embodiments. In another alternative, as part of an eleventh embodiment, the anesthetic of the disclosed compositions is delivered to the eye in a sustained manner for a period ranging from about 12 hours to about 2 days, wherein the remaining features of the compositions are described herein e.g., as in the first through tenth embodiments. In another alternative, as part of an eleventh embodiment, the anesthetic of the disclosed compositions is delivered to the eye in a sustained manner for a period ranging from about 18 hours to about 10 days, 18 hours to about 9 days, 18 hours to about 8 days, 18 hours to about 7 days, 18 hours to about 6 days, 18 hours to about 5.5 days, 18 hours to about 5 days, about 18 hours to about 4.5 days, 18 hours to about 4 days, about 18 hours to about 3.5 days, 18 hours to about 3 days, about 18 hours to about 2.5 days, 18 hours to about 2 days, about 24 hours to about 10 days, 24 hours to about 9 days, 24 hours to about 8 days, 24 hours to about 7 days, 24 hours to about 6 days, 24 hours to about 5.5 days, 24 hours to about 5 days, about 24 hours to about 4.5 days, 24 hours to about 4 days, about 24 hours to about 3.5 days, 24 hours to about 3 days, about 24 hours to about 2.5 days, 24 hours to about 2 days, or for about 24 hours, about 36 hours, about 2 days, about 2.5 days, about 3 days, about 3.5 days, about 4 days, about 4.5 days, about 5 days, about 5.5 days, about 6 days, about 6.5 days, about 7 days, about 7.5 days, about 8 days, about 8.5 days, about 9 days, about 9.5 days, or about 10 days, wherein the remaining features of the compositions are described herein e.g., as in the first through tenth embodiments.

As part of a twelfth embodiment, the anesthetic in the disclosed composition is microencapsulated, wherein the remaining features of the compositions are described herein e.g., as in the first through eleventh embodiments.

As part of a thirteenth embodiment, the anesthetic in the disclosed composition is microencapsulated with poly(lactic-co-glycolic acid) (PLGA) or poly(lactic acid) (PLA), or a combination thereof, wherein the remaining features of the compositions are described herein e.g., as in the first through twelfth embodiments. Alternatively, as part of a thirteenth embodiment, the anesthetic in the disclosed composition is microencapsulated with PLGA, wherein the remaining features of the compositions are described herein e.g., as in the first through twelfth embodiments.

Anesthetics that can be used in the composition described herein include those that are suitable for ocular use. As part of a fourteenth embodiment, the anesthetic in the disclosed compositions is selected from bupivacaine, lidocaine, proparacaine, tetracaine, dibucaine, benoxinate, ropivacaine, articaine, carbocaine, marcaine, mepivacaine, polocaine, prilocaine, sensorcaine, and septocaine, wherein the remaining features of the compositions are described herein e.g., as in the first through thirteenth embodiments. Alternatively, as part of a fourteenth embodiment, the anesthetic in the disclosed compositions is selected from bupivacaine, lidocaine, proparacaine, and tetracaine, wherein the remaining features of the compositions are described herein e.g., as in the first through thirteenth embodiments. In another alternative, as part of a fourteenth embodiment, the anesthetic of the compositions described herein is bupivacaine, wherein the remaining features of the compositions are described herein e.g., as in the first through thirteenth embodiments.

As part of a fifteenth embodiment, the hydrogel compositions described herein comprise a clearance zone that is devoid of the anesthetic (e.g., undissolved anesthetic) prior to release of the anesthetic, wherein the remaining features of the compositions are described herein e.g., as in the first through fourteenth embodiments. By way of example, in one aspect of this embodiment, particulate anesthetic is comprised in the polymer network of the hydrogel, but is not present in the clearance zone. In one aspect, based on the design and properties of the polymer network, only the dissolved anesthetic passes through the clearance zone and out of the hydrogel and into the eye.

As part of a sixteenth embodiment, the anesthetic in the compositions described herein is present in the hydrogel composition at or near its saturation level, wherein the remaining features of the compositions are described herein e.g., as in the first through fifteenth embodiments.

As part of a seventeenth embodiment, the hydrogel compositions described herein comprise a clearance zone, wherein the size of the clearance zone increases as a function of the amount of anesthetic release, wherein the remaining features of the compositions are described herein e.g., as in the first through sixteenth embodiments.

As part of an eighteenth embodiment, the hydrogel compositions described herein are in the form of an intracanalicular insert, wherein the remaining features of the compositions are described herein e.g., as in the first through seventeenth embodiments.

As part of a nineteenth embodiment, the hydrogel compositions described herein are in the form of an insert for delivery to the fornix of the eye, wherein the remaining features of the compositions are described herein e.g., as in the first through eighteenth embodiments.

As part of a twentieth embodiment, the hydrogel composition is fully degraded following complete release of said anesthetic, wherein the remaining features of the compositions are described herein e.g., as in the first through nineteenth embodiments. Alternatively, as part of an twentieth embodiment, the hydrogel implant is fully degraded after about 12 months, after about 11 months, after about 10 months, after about 9 months, after about 8 months, after about 6 months, after about 5 months, after about 4 months, after about 3 months, after about 2 months, after about 1 month (i.e., after about 30 days) following complete release of the anesthetic, wherein the remaining features of the compositions are described herein e.g., as in the first through nineteenth embodiments. Alternatively, as part of an twentieth embodiment, the hydrogel implant is fully degraded following at least 90% (e.g., at least 91%, at least 92%, at least 93%, at least 94%, at least 95%, at least 96%, at least 97%, at least 98%, or at least 99%) release of the anesthetic, wherein the remaining features of the compositions are described herein e.g., as in the first through nineteenth embodiments.

As part of a twenty-first embodiment, the hydrogel composition further comprises fluorescein, wherein the remaining features of the compositions are described herein e.g., as in the first through twentieth embodiments.

### Methods, Processes, and Use

The disclosed hydrogel compositions are useful in treating and preventing ocular discomfort. Thus, provided herein are methods of treating or preventing ocular discomfort in a subject comprising administering to the subject an effective amount of a composition described herein. Also disclosed in the use of a disclosed composition for treating or preventing ocular discomfort in a subject. Further provided is the use of a disclosed composition in the manufacture of a medicament for treating or preventing ocular discomfort.

Ocular discomfort includes instances where there is a lack of ease in or about the eye or eyes. This includes e.g., foreign body sensations such as gritty, sandy, and granular sensation (upon blinking), feeling something in the eye, feels as if there is a grain of sand or eyelash in the eye; burning sensation; stinging of the eye; irritation; soreness; dryness; itching or scratchiness (e.g., cause by allergic reaction); pain such as aching, eye strain, deep/dull (orbital/brow) pain, heaviness, headache around the eye, sharp pain, stabbing sensation, sharp pin, throbbing, beating, pulsating, pain on movement, and tenderness (to touch); fatigue associated conditions such as tiredness, need/desire to close, bother when open and close the eyes, and feel more comfortable with the eyes closed; sensitivity reactions e.g., photosensitivity, sensitivity to wind; discharges such as secretion, tearing, watering, discharge (ropy), mucus, crusting; autonomic symptoms such as heat, warmth, coldness; pain with eye movements; and general redness, tingling, and blinking. Ocular discomforts can also be caused by trauma, infection, inflammation, or surgery.

In one aspect, the ocular discomfort treated or prevented herein is pain. In another aspect, the ocular discomfort treated or prevented herein is pain caused by surgery. In another aspect, the ocular discomfort treated or prevented herein is post-ocular injection pain. In another aspect, the ocular discomfort treated or prevented herein is a corneal abrasion or trauma. In another aspect, the ocular discomfort treated or prevented herein is caused by an ocular inflammatory condition.

### EXEMPLIFICATION

### 1. Materials and Methods

Bupivacaine microspheres were produced using bupivacaine free base (BFB) (Spectrum Chemical, Part#: B2353) and PLGA (Sigma-Aldrich, PN: 719897, Resomer RG 502 H). BFB (814 mg) and PLGA (804 mg) were mixed and dissolved in dichloromethane (3.155 g) (Sigma Aldrich, SHBH9222) to create the dispersed phase (DP). The continuous phase (CP) (500mL) contained 0.5% polyvinyl alcohol (Spectrum Chemical, 2GK0231), 2.5% sodium chloride (Spectrum Chemical, 1F10675), saturated with BFB, and adjusted to pH 10.5 using 1M potassium phosphate tribasic (Sigma Aldrich, MKCF3247). The CP (500 mL) was added to a jacketed reactor (500 mL, Wilmad Lab Glass, LG-8079B-100) equilibrated to 5° C. stirred at 900 rpm. The DP was injected into the CP at a rate of 350 μL/min through a 23G needle using a syringe pump. The final volume ratio of DP to CP was 1:100. The temperature ramp profile following injection was 5° C. for 20 mins, 20° C. for 1 hr, and then 30° C. for 2 hrs. The hardened microspheres were then harvested and fractionated on sieves (20-53 μm) while washing with ample water (7 L of 20-25° C. RODI water) to remove CP. The microspheres were then transferred to glass vials (10 mL) and lyophilized to dry. Based on weight calculations of starting materials the final yield was estimated at 10% with an estimated drug encapsulation efficiency of 96%.

BFB-PLGA microspheres were mixed with hydrogel precursors, PEG ester (4a20k SG, JenKem, C53-100801) and trilysine acetate salt (Bachem, 08-025) with pH modifying agents (sodium phosphate mono and dibasic) to achieve a 14% PEG concentration (wet weight) and 20% microspheres concentration (wet weight) in the formulation. Prior to gelation, the mixed formulation was cast into 1.3 mm ID silicone tubing (Cole Parmer) and cured for 1 hour at ambient temperature. The formulated reacted hydrogel in the tubing at lengths of 16 cm was then stretched 2.5× in a stretcher and dried in a glove bag under nitrogen for 72 hours. The dried bupivacaine/PLGA/hydrogel strands were then removed and cut to 3 mm lengths. The cut inserts were packaged in 10 mL glass scintillation vials under dried nitrogen, and then sealed in foil pouches. They were then sterilized via gamma irradiation at 28.5 to 34.8 kGy. Table 1 shows the normalized formulated biodegradable ocular hydrogel composition comprising bupivacaine (“Inventive Composition”).

TABLE 1
Formulated Composition and Dimensions

| Component | % Dry Basis (w/w) |
|---|---|
| 4a20k PEG SG | 38% |
| Trilysine acetate | 1% |
| Resomer RG 502 H | 29% |
| Bupivacaine Free Base | 27% |
| Sodium Phosphate Monobasic | 1% |
| Sodium Phosphate Dibasic | 3% |
| Dimensions | Diameter and Length |
| Dry | 0.5 mm × 3.0 mm |
| Hydrated | 1.8 mm × 2.2 mm |

2. Study Design on Male Beagle Dogs with Corneal Wounds

Prior to treatment, all beagles received a clinical ophthalmic examination for baseline observations. Seventeen beagle dogs were split into three groups, as shown in Table 2.

TABLE 2
Inventive Composition Nonclinical Study Design

| | | | | | Slit-lamp | | |
|---|---|---|---|---|---|---|---|
| | | | | | Imaging and | | |
| | | Treatment | | | Fluorescein | Tear | Corneal |
| Group | N | OD | OS | Dose Route | Staining | Collection | Esthesiometry |
| 1 | 5 | Control | IC | Intracanalicular | Days | Days | Acclimation and Days 0, |
| | | (no insert) | | Insertion | 0, 3, 5, 7 | 3, 4, 5 | 3-7, 10-14, 31-35, 38-42 |
| 2 | 5 | | | on Day 0 | Days | Days | Acclimation and Days 0- |
| | | | | | 0, 2, 4, 7 | 1,2, 3,4 | 4,7-11, 14, 28-32, 35-39 |
| 3 | 7 | Untreated | | NA | NA | NA | Acclimation and Days 1- |
| | | | | | | | 3, 6-7 |

Corneal wounds were created in both eyes (OU) of ten female beagle dogs on Day 0 using epithelial debridement. Animals were post-operatively monitored and treated with eye drops. The Inventive Composition (IC) containing approximately 160 μg of bupivacaine was inserted into the lower or upper lid punctum of one eye of all ten dogs on Day 0 after corneal wounding and removed on Day 7 after wounding. Clinical ophthalmic examinations (slit-lamp biomicroscopy) were conducted daily on weekdays through Day 7. Fluorescein staining was performed and slit-lamp photographs were taken on Days 0, 3, 5, and 7 (Group 1) or on Days 0, 2, 4, and 7 (Group 2). Corneal esthesiometry was performed at baseline and on Days 0, 3-7, 10-14, 31-35, and 38-42 (Group 1) or on Days 0-4, 7-11, 14, 28-32, and 35-39 (Group 2). General health observations and gross ocular observations were performed daily from Day 1 through Day 11 (Group 1) or Day 8 (Group 2). Body weights were recorded prior to dosing and on Day 7. Tears were collected on Days 3, 4, and 5 (Group 1) or on Days 1, 2, 3, and 4 (Group 2). The two groups were staggered in order to collect tear film samples on days 1, 2, 3, 4 and 5 using Schirmer test strips. Tear film samples were collected in the morning prior to administration of drops, in order to avoid dilution of the samples. Pre and post weights were collected on the tear film strips, and samples were sent to PharmOptima, LLC (Portage, Mich.) for bioanalysis via LC/MS.

In seven additional, untreated (no corneal wounding and no Inventive Composition inserts) female beagle dogs (Group 3), corneal esthesiometry was performed for 7 days (2 acclimation days, Days 1-3, and Days 6-7).

### 3. Results and Discussion

A. Pharmacokinetics and In Vitro Release

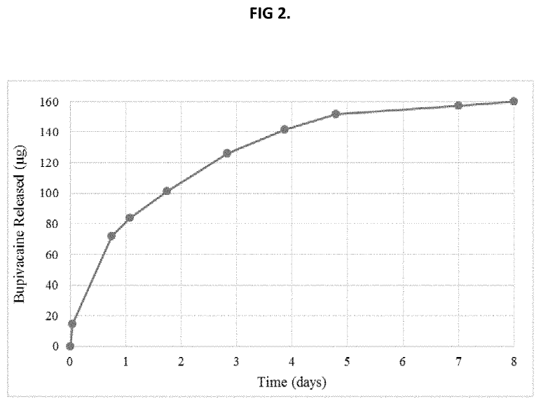

The PK portion of the study measured concentrations of bupivacaine released from the Inventive Composition into the tear fluid over 5 days following intracanalicular administration in beagle dogs and results are presented in Table 3. Tear fluid samples were collected pre-drop administration to prevent dilution of the bupivacaine concentrations. The PK profile indicates elevated concentrations of bupivacaine in the tear fluid through 4 days with a decrease observed at 5 days. The decrease in bupivacaine concentrations at 5 days corresponds to the in vitro release performed in physiological relevant media (PBS, pH 7.4 at 37° C.) that demonstrated near complete bupivacaine release from the Inventive Composition by 5 days, as seen in FIG. 2. Bupivacaine release rates that were calculated on an hourly basis from the in vitro testing analysis are listed in Table 4. Results demonstrates a maximal bupivacaine release of 14.6 μg/hour during the burst phase (0 to 1 hour) following placement in dissolution media and then a tapering of bupivacaine released over the first 5 days of sampling with minimal drug release between 5 and 8 days.

TABLE 3
Bupivacaine Concentrations in Beagle
Tear Fluid Over 5 Days (Groups 1 and 2)

| | | Average | Std. Dev. | Minimum | Median | Maximum |
|---|---|---|---|---|---|---|
| Day | N = | (μg/mL) | (μg/mL) | (μg/mL) | (μg/mL) | (μg/mL) |
| 1 | 3 | 0.78 | 0.65 | 0.28 | 0.55 | 1.52 |
| 2 | 3 | 0.38 | 0.34 | 0.15 | 0.23 | 0.77 |
| 3 | 6 | 0.74 | 0.61 | 0.10 | 0.59 | 1.52 |
| 4 | 6 | 0.57 | 0.49 | 0.08 | 0.45 | 1.18 |
| 5 | 3 | 0.25 | 0.17 | 0.10 | 0.23 | 0.43 |

TABLE 4
Bupivacaine Release Rates from In Vitro Testing

| In Vitro Sampling | Bupivacaine |
|---|---|
| Period | Release Rate |
| (hours) | (μg/hour) |
| 0-1 | 14.6 |
| 1-18 | 3.4 |
| 18-26 | 1.5 |
| 26-42 | 1.1 |
| 42-68 | 1.0 |
| 68-93 | 0.6 |
| 93-115 | 0.4 |
| 115-168 | 0.1 |

B. Pharmacodynamic Performance

Corneal sensitivity was used as a measure of pharmacodynamic performance. It was recorded using a Cochet-Bonnet esthesiometer, a nylon filament that is designed to incur a force on the cornea that elicits a reflexive reaction from the dog, exhibited in the form of a blink or physical withdrawal. The length of the filament at the time of this reaction is the score recorded. The lower this score is, the more force required to elicit a reaction (shorter filament length). This force increases exponentially as the filament becomes shorter.

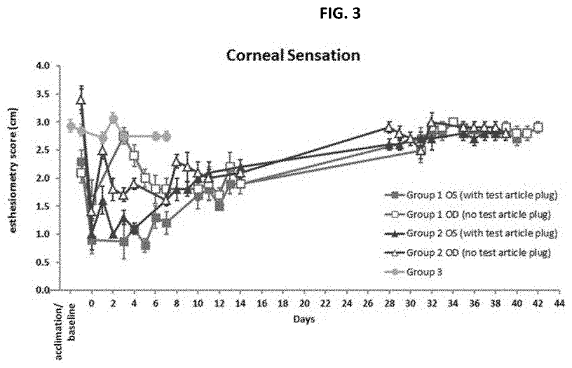

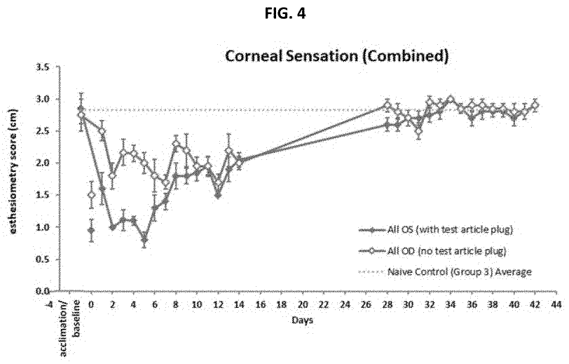

Corneal sensitivity was compared between the test article treated animals (Groups 1 and 2 OS; Inventive Composition treated plus standard of care following PRK), control animals (Groups 1 and 2 OD: standard of care following PRK) and naïve control Group 3 (OU; untreated) and average results with standard error of the mean (error bars) are plotted in FIG. 3 and combined average results for the test article, untreated and naïve eyes are plotted in FIG. 4

On Day 0 shortly after corneal wounding and test article administration, corneal sensation was sharply decreased both in eyes that had not received a test article insert (“untreated eyes”) and in test article insert-treated eyes, with a trend towards a greater decrease in test article-treated eyes. On the day after corneal wounding and test article administration (Day 1), corneal sensation was near baseline in untreated eyes and slightly decreased from baseline in test article-treated eyes. Starting on Day 2 and continuing through Day 7, untreated eyes exhibited a moderate decrease in corneal sensation compared to baseline levels and to average corneal sensation levels in naïve controls (Group 3), while test article-treated eyes exhibited a substantially greater decrease. After Day 7 (the day of test article removal), corneal sensation in test article-treated eyes increased and became comparable to untreated eyes at all later time points. Corneal sensation continued to be moderately decreased from baseline for a second week after corneal wounding (through Day 14) in both treated and untreated wounded eyes. By four weeks after corneal wounding (starting Day 28), corneal sensation had returned to baseline levels in both treated and untreated wounded eyes and was comparable to average corneal sensation levels in naïve controls. Corneal sensation in naïve controls (Group 3) remained stable at all evaluated time points.

Esthesiometry showed a moderate reduction in corneal sensation compared to baseline, as well as to average corneal sensation in naïve (no corneal wounding and no Inventive Composition inserts) control eyes, and in wounded eyes without Inventive Composition inserts. This decrease was noted starting two days after corneal wounding and lasted through two weeks after corneal wounding. Decreased corneal sensation after corneal de-epithelization has been documented in both rabbits and humans, and reduced corneal sensation after corneal de-epithelization has been found to be associated with sensory denervation of the cornea. See e.g., Babst, C. R. and Gilling, B. N. Bupivacaine: A Review. Anesth. Prog. 25(3), 87-91 (1978).

A substantially greater reduction in corneal sensation was seen in wounded eyes treated with Inventive Composition during the first week after corneal wounding (FIGS. 3 and 4) compared to untreated eyes. This difference was only observed while the Inventive Composition insert was present and after removal of Inventive Composition on Day 7, corneal sensation in Inventive Composition

treated eyes returned to levels comparable to the untreated wounded eyes. Like wounded eyes without Inventive Composition inserts, Inventive Composition treated eyes whose insert had been removed exhibited moderate reduction in corneal sensation lasting through two weeks after corneal wounding, likely reflecting sensory denervation of the cornea subsequent to corneal de-epithelization (as mentioned above).

By four weeks after corneal wounding, the decrease in corneal sensation had resolved in all eyes, likely reflecting epithelial reinnervation that followed regeneration of the corneal epithelium after wound healing. Such reinnervation of the corneal epithelium, and its association with recovery of corneal sensation, has been documented during recovery from corneal deepithelialization in rabbits (deLeeuw A, Chan K. Corneal nerve regeneration: correlation between morphology and restoration of sensitivity. Invest Ophthalmol Vis Sci. 1989; 30:1980-1990) and humans (Campos M et al. Corneal sensitivity after photorefractive keratectomy. Am J Ophthalmol. 1992 Jul. 15; 114(1):51-4).

C. Safety and Corneal Wound Healing

No effects of Inventive Composition administration on general health were observed. Ophthalmic slit-lamp examinations were performed on Groups 1 and 2, and not performed on Group 3 (naïve control). After corneal epithelial debridement, all wounded eyes exhibited ocular irritation, characterized by ocular hyperemia/conjunctival congestion, swelling, and/or discharge. These symptoms resolved in all eyes over the course of the first week after wounding. All wounded eyes also exhibited corneal opacity after epithelial debridement. Corneal opacity was observed in all eyes up to the first four days after wounding, which resolved in some eyes while persisting in others through the final ophthalmic examination 7 days after wounding. One wounded eye also developed corneal edema. Immediately after epithelial debridement, pupillary response was also impaired in some eyes, and one eye exhibited anterior lens opacity. These findings all resolved by the next day.

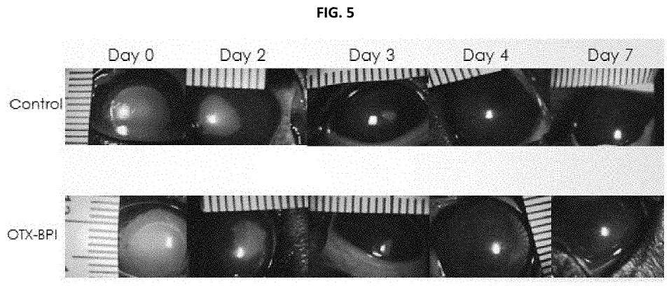

Eyes treated with the Inventive Composition insert and eyes without an insert did not differ in how much they were affected by any of these ocular symptoms. Mild to substantial weight loss was seen in all animals that had undergone corneal wounding and it was likely due to the collars placed on these animals interfering with feeding. Fluorescein staining was performed to measure wound size and healing. Injured tissue is quantified by staining with fluorescein and imaging the cornea under blue light over time, as seen in FIG. 5. Injured tissue will glow due to absorption of the fluorescein stain, and this can be quantified using imaging software.

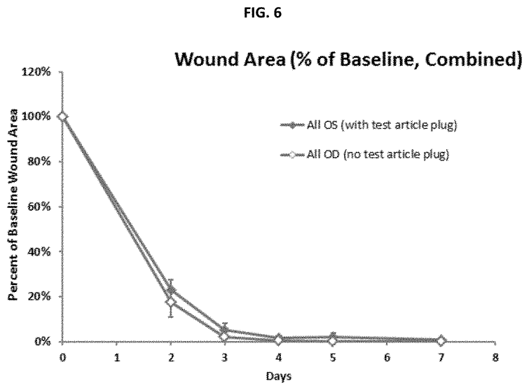

The corneal wound area diminished rapidly in most eyes, with several eyes exhibiting no measurable wound area as early as 2 to 3 days after wounding, and 19 of 20 wounded eyes exhibiting no measurable wound area within 7 days after wounding. There was no substantial difference in the rate of wound healing between eyes treated and not treated with the Inventive Composition insert (FIG. 6).

While have described a number of embodiments of this, it is apparent that our basic examples may be altered to provide other embodiments that utilize the compounds and methods of this disclosure. Therefore, it will be appreciated that the scope of this disclosure is to be defined by the appended claims rather than by the specific embodiments that have been represented by way of example.

1. A biodegradable ocular hydrogel composition comprising an anesthetic and a polymer network, wherein said anesthetic is delivered to the eye in a sustained manner for a period of about 12 hours or longer.

2. The hydrogel composition of claim 1, wherein the polymer network comprises a plurality of polyethylene glycol (PEG) units.

3. The hydrogel composition of claim 1, wherein the polymer network comprises a plurality of multi-arm PEG units having from 2 to 10 arms.

4. The hydrogel composition of claim 1, wherein the polymer network comprises a plurality of multi-arm PEG units having from 4 to 10 arms.

5. The hydrogel composition of claim 1, wherein the polymer network comprises a plurality of multi-arm PEG units having from 4 to 8 arms.

6. The hydrogel composition of claim 1, wherein the polymer network comprises a plurality of multi-arm PEG units having 8 arms.

7. The hydrogel composition of claim 1, wherein the polymer network comprises a plurality of multi-arm PEG units having 4 arms.

8. The hydrogel composition of claim 1, wherein the polymer network comprises a plurality of PEG units having the formula: 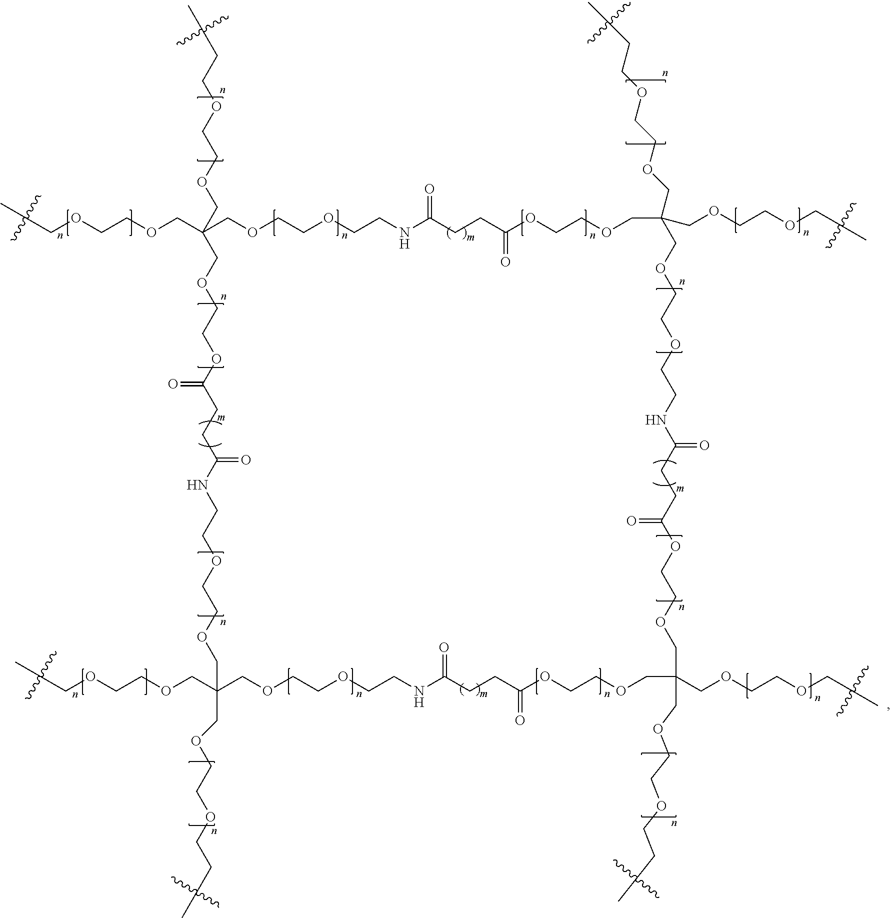
wherein n represents an ethylene oxide repeating unit and the dashed lines represent the points of repeating units of the polymer network

9. The hydrogel composition of claim 1, wherein the polymer network is formed by reacting a plurality of polyethylene glycol (PEG) units selected from 4a20K PEG SAZ, 4a20K PEG SAP, 4a20K PEG SG, 4a20K PEG SS, 8a20K PEG SAZ, 8a20K PEG SAP, 8a20K PEG SG, 8a20K PEG SS with one or more PEG or Lysine based-amine groups selected from 4a20K PEG NH2, 8a20K PEG NH2, and trilysine, or a salt thereof.

10. The hydrogel composition of claim 1, wherein the polymer network is formed by reacting 4a20k PEG SG with trilysine or a salt thereof.

11. The hydrogel composition of claim 1, wherein the polymer network is amorphous under aqueous conditions.

12. The hydrogel composition of claim 1, wherein the polymer network is semi-crystalline in the absence of water.

13. The hydrogel composition of claim 1, wherein the particulate anesthetic inhibitor is homogenously dispersed within the polymer network.

14. The hydrogel composition of claim 1, wherein the anesthetic is delivered to the eye in a sustained manner for a period ranging from about 12 hours to about 10 days.

15. The hydrogel composition of claim 1, wherein the anesthetic is delivered to the eye in a sustained manner for a period for a period ranging from about 12 hours to about 7 days.

16. The hydrogel composition of claim 1, wherein the anesthetic is delivered to the eye in a sustained manner for a period for a period ranging from about 12 hours to about 4 days.

17. The hydrogel composition of claim 1, wherein the anesthetic is delivered to the eye in a sustained manner for a period ranging from about 18 hours to about 4 days, about 24 hours to about 4 days, 12 hours to about 3.5 days, 18 hours to about 3.5 days, 24 hours to about 3.5 days, 12 hours to about 3 days, 18 hours to about 3 days, 24 hours to about 3 days, 12 hours to about 2.5 days, 18 hours to about 2.5 days, 24 hours to about 2.5 days, 12 hours to about 2 days, 18 hours to about 2 days, 24 hours to about 2 days; or for about 24 hours, about 36 hours, about 2 days, about 2.5 days, about 3 days, about 3.5 days, or about 4 days.

18. The hydrogel composition of claim 1, wherein the anesthetic is microencapsulated.

19. The hydrogel composition of claim 1, wherein the anesthetic is microencapsulated with poly(lactic-co-glycolic acid) (PLGA) or poly(lactic acid) (PLA), or a combination thereof.

20. The hydrogel composition of claim 1, wherein the anesthetic is microencapsulated with PLGA.

21. The hydrogel composition of claim 1, wherein the anesthetic is selected from bupivacaine, lidocaine, proparacaine, tetracaine, dibucaine, benoxinate, ropivacaine, articaine, carbocaine, marcaine, mepivacaine, polocaine, prilocaine, sensorcaine, and septocaine.

22. The hydrogel composition of claim 1, wherein the anesthetic is selected from bupivacaine, lidocaine, proparacaine, and tetracaine.

23. The hydrogel composition of claim 1, wherein the anesthetic is bupivacaine.

24. The hydrogel composition of claim 1, wherein the hydrogel composition comprises a clearance zone that is devoid of the undissolved anesthetic prior to release of the anesthetic.

25. The hydrogel composition of claim 1, wherein the anesthetic is present in the hydrogel composition at or near its saturation level.

26. The hydrogel composition of claim 1, wherein the size of the clearance zone increases as a function of the amount of anesthetic release.

27. The hydrogel composition of claim 1, wherein the hydrogel composition is an intracanalicular insert.

28. The hydrogel composition of claim 1, wherein the hydrogel composition is for delivery to the fornix of the eye.

29. The ocular insert or insert of claim 1, wherein the hydrogel composition is fully degraded following release of the anesthetic.

30. A method of treating or preventing ocular discomfort in a subject, comprising administering to the eye of the subject a therapeutically effective amount of the hydrogel composition of claim 1.

31. The method of claim 30, wherein the ocular discomfort is caused by trauma, drying, infection, inflammation, surgery, irritation, or itching.
